(19) United States
(12) Patent Application Publication
(10) Pub. No.: US 2005/0282273 A1
(43) Pub. Date: Dec. 22, 2005

(54) Medium and methods for culturing of avian primordial germ cells

(76) Inventor: Donald W. Swiatek II, Candia, NH (US)

Correspondence Address: Judy Jarecki-Black; Ph.D, J.D, 3239 Satellite Blvd., Duluth, GA, 30096, US

(21) Appl. No.: 11/148,039

(22) Filed: Jun. 8, 2005

Related U.S. Application Data
(60) Provisional application No. 60/578,537, filed on Jun. 10, 2004.

(51) Int. Cl. C12N 5/08

(52) U.S. Cl. 435/366; 435/404

(57) ABSTRACT

The invention provides a novel culture medium useful for the proliferation and maintenance of avian primordial germ cells (PGCs) and encompassing a medium base, leukemia inhibitory factor, basic fibroblast growth factor, stem cell factor and insulin-like growth factor, and an avian serum. The invention also provides a method for the proliferation and maintenance of avian primordial germ cells for extended periods encompassing the steps of isolating a population of PGCs from an avian and culturing the PGCs in a culture medium containing the growth factors and avian serum. The invention further provides methods of producing chimeric avians by isolating and culturing PGCs in a culture medium containing avian serum, transferring the PGCs into a recipient avian embryo, and allowing the recipient avian to develop into a chimeric bird. Another aspect of the invention provides a culture of avian PGCs grown and maintained in the culture medium containing avian serum.

### CROSS-REFERENCE TO RELATED APPLICATIONS

This application claims priority from U.S. Provisional Patent Application Ser. No. 60/578,537 filed Jun. 10, 2004 and incorporated herein by reference in its entirety.

### INCORPORATION BY REFERENCE

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to an improved cell culture medium and method for the proliferation and maintenance of avian cells, in particular primordial germ cells (PGCs), and most particularly chicken PGCs, for prolonged periods in tissue culture. The invention further relates to the use of PGCs cultured with the improved medium and which may be modified by the insertion of desired DNA sequences to produce chimeric birds, in particular chimeric chickens.

### BACKGROUND OF THE INVENTION

The success of embryonic stem (ES) cell technology for the production of “knock-out” mice has led to research focused toward the development of tissue culture systems for ES cells and primordial germ cells (PGCs) in farm animal species. The ability to maintain ES undifferentiated cells in continuous culture enables in vitro transfection of such cells and ideally the selection of transfected cells that contain a desired gene prior to their transfer to the inner cell mass of a developing embryo to generate chimeric animals. Ideally, at least some of the resultant chimeric animals will be able to segregate the DNA construct via the germ line and hence to produce transgenic progeny.

There has been considerable research targeted toward modifying the genome of Gallinacea and chickens in particular because of the considerable economic importance thereof. There are basically two alternative routes under investigation for producing transgenic chickens. The two routes are distinguished on the basis of the time when manipulation of the genome is effected, i.e., before lay or after lay. The latter method includes the transfer of donor ES cells and PGCs to recipient embryos. In both routes, the bulk of the work has been effected by infecting donor cells with retroviral vectors containing a gene of interest.

The approach that comprises manipulation of the genome after lay has been successful. Chimeric birds generated by the injection of laid eggs with replication competent retroviral vectors have shown germ line transmission to between 1% and 11% of their offspring. Injection of replication-defective retroviral vectors into laid eggs has generated chimeric male birds that transmitted the vector to their offspring.

A significant problem with all of these methods is that long-term culture systems for chicken ES cells and PGCs have been relatively difficult to establish. Previous PGC culturing methods have included the use of growth factors, in particular leukemic inhibitory factor (LIF) or insulin-like growth factor (IGF). However, such methods have not been able to provide prolonged culturing periods, a prevalent concern as it would facilitate the production of transgenic PGCs.

Pain et al., (1996) Development, 122: 2329-2398, have demonstrated putative chicken ES cells obtained from blastodermal cells. They further reported maintenance of these cells in cultures for 35 passages supposedly without loss of the ES phenotype as defined by monoclonal antibodies specific for mouse ES cells. These putative ES cells apparently developed into PGCs upon transfer into avian embryos where they colonized in the gonads. However, the authors did not definitively establish that these cells were in fact ES cells.

The cross-reactivity of mouse ES cell-specific monoclonal antibodies with chicken ES cells argues for conservation of ES cell receptors across species. Also, the fact that it was also able to generate two chimeric chickens with injections of 7-day-old blastodermal cell cultures would suggest the presence of ES cells in the system. However, these researchers did not rule out the possibility that PGCs were present in their complex culture system.

An alternative route to the production of ES cells comprises PGCs. Procedures for the isolation and transfer of PGCs from donor to recipient embryos have been developed and have successfully led to the generation of chimeric chickens with germ line transmission of the donor genotype (Vick et al., (1993) Proc. Royal Soc. London Ser. B, 251: 179-182; Tajima et al., (1993) Theriogenology, 40: 509-519). Further, PGCs have been cryopreserved and later thawed to generate chimeric birds (Naito et. al., 1994) J. Reprod. Fertil., 102: 321-325). However, this system is very labor intensive and yields, on average, only about 50 to 80 PGCs per embryo. Infection of PGCs with retroviral vectors has also been reported. It is clear that improved methods for culturing PGCs comprises a significant need in the art.

The growth of avian, and especially of chicken, ES cells and PGCs has been achieved as disclosed in U.S. Pat. No. 6,156,569 to Ponce de Leon et al. incorporated herein by reference in its entirety. However, prolonged culturing of PGCs in known media still results in extensive cell death as evidenced by frequent cell debris formation. There exists a need, therefore, for a culture medium more suitable for promoting the proliferaion and maitenance of avian ES cells and PGCs under in vitro conditions.

Citation or identification of any document in this application is not an admission that such document is available as prior art to the present invention.

### SUMMARY OF THE INVENTION

The present invention provides a novel medium and method for maintaining avian (advantageously chicken) primordial germ cells (PGCs) in tissue culture for prolonged periods, i.e., for at least 25 days, more advantageously at least about 50 days, and ideally indefinitely.

Present methods for maintaining avian PGCs in tissue culture reliably provide for their maintenance for not much more than about 25 days (as demonstrated by their ability to produce chimeric avians). The present invention encompasses modified and improved culture media comprising at least the growth factors leukemia inhibitory factor (LIF), basic fibroblast growth factor (bFGF), stem cell factor (SCF) and insulin-like growth factor (IGF) and an avian serum, which enable avian primordial germ cells, especially chicken primordial germ cells to be maintained and to proliferate at a faster rate and for more prolonged periods, i.e., at least 25 days, and to be maintained for substantially longer periods in tissue culture than is the case with currently used media.

The medium of the present invention supports an improved rate of PGC growth. There are also substantial improvements in the prolongation of viability of PGC cultures to at least about 50 days duration. The PGCs cultured in the novel medium for at least about 50 days retain PGC characteristics such as being positive for Periodic Acid Schiff staining and PGC-specific antigens. Injection of such cells into chicken embryos results in incorporation of the PGCs into the gonads. Moreover, these PGCs have been demonstrated to be useful for the generation of chimeric chickens.

The PGCs cultured using the media of the present invention may be useful for the production of transgenic avian PGCs, which can be used to produce transgenic chimeric avians. It is expected that these transgenic chimeric avians will be useful for recovery of heterologous proteins, which advantageously can be recovered directly from the eggs of such chimeric transgenic avians. For example, such avians can be used for the production and recovery of therapeutic proteins and other polypeptides.

One aspect of the invention, therefore, provides a modified culture medium for the proliferation and maintenance of avian primordial germ cells, the medium comprising in a medium base, the growth factors leukemia inhibitory factor (LIF), basic fibroblast growth factor (bFGF), stem cell factor (SCF) and insulin-like growth factor (IGF), and avian serum.

In the various embodiments of the invention, the avian serum may be present in the medium at a concentration of between about 0.5% v/v and about 10% v/v. In certain embodiments, the avian serum may be present in the medium at a concentration of between about 1% v/v and about 6% v/v. In one advantageous embodiment of the invention, the avian serum may be present in the medium at a concentration of about 5% v/v. In various embodiments of the invention, the avian serum may be present in the medium at a concentration of about 1% v/v.

In the various embodiments of the invention, the avian serum may be from any avian source. In a particularly advantageous embodiment of the invention, however, the avian serum may be chicken serum or turkey serum and most advantageously, chicken serum.

In the various embodiments of the invention, the culture medium for the proliferation and maintenance of avian primordial germ cells may contain in a medium base leukemia inhibitory factor (LIF) between about 0.00625 U/μl and about 5 U/μl, basic fibroblast growth factor (bFGF) between about 0.25 pg/μl and about 100 pg/μl, stem cell factor (SCF) between about 4 pg/μl and about 200 pg/μl and insulin-like growth factor (IGF) between about 0.5625 pg/μl and about 150 pg/μl.

In some embodiments of this aspect of the invention, the leukemia inhibitory factor (LIF) may be between about 0.25 U/μl and about 3 U/μl, basic fibroblast growth factor (bFGF) is between about 25 pg/μl and about 100 pg/μl, stem cell factor (SCF) may be between about 40 pg/μl and about 200 pg/μl and insulin-like growth factor (IGF) may be between about 10 pg/μl and about 150 pg/μl.

In an advantageous embodiment of the invention, the leukemia inhibitory factor (LIF) may be about 1 U/μl, basic fibroblast growth factor (bFGF) may be about 40 pg/μl, stem cell factor (SCF) may be about 80 pg/μl and insulin-like growth factor (IGF) may be about 60 pg/μl.

In the various embodiments of the invention, the culture medium may further comprise 2-β-mercaptoethanol having a concentration of between about 0.05 mM and about 50 mM. In one embodiment of the invention, the 2-β-mercaptoethanol may have a concentration of between about 0.05 mM and about 0.25 mM. In one advantageous embodiment, the 2-β-mercaptoethanol may have a concentration of about 0.13 mM.

This aspect of the invention also encompasses a culture medium for the proliferation and maintenance of avian primordial germ cells which may comprise a medium, fetal bovine serum, avian serum, L-glutamine, an antibiotic, 2-β-mercaptoethanol, leukemia inhibitory factor (LIF), basic fibroblast growth factor (bFGF), stem cell factor (SCF) and insulin-like growth factor (IGF).

In a particularly advantageous embodiment of the invention, the culture medium may comprise a medium, about 10% fetal bovine serum, about 5% chicken serum, about 2 mM L-glutamine, about 1% antibiotic, about 0.13 mM 2-β-mercaptoethanol, about 1 U/μl leukemia inhibitory factor (LIF), about 40 pg/μl basic fibroblast growth factor (bFGF), about 80 pg/μl stem cell factor (SCF) and about 60 pg/μl insulin-like growth factor (IGF).

Another aspect of the invention encompasses a culturing method for the proliferation and maintenance of avian primordial germ cells for periods of at least 25 days in tissue culture, the method may comprise isolating a pure population of primordial germ cells from a desired avian, and culturing the isolated, pure population of primordial germ cells (PGCs) in a culture medium which may comprise leukemia inhibitory factor (LIF), basic fibroblast growth factor (bFGF), stem cell factor (SCF) and insulin-like growth factor (IGF), and avian serum.

In the various embodiments of this aspect of the invention, the avian PGCs may be obtained from an avian of the genus Gallinacea. In one embodiment of the invention, the PGCs may be chicken PGCs or turkey PGCs.

Yet another aspect of the invention provides a method of producing chimeric avians, the method may comprise isolating a pure population of primordial germ cells from a desired avian; maintaining the isolated pure population of primordial germ cells (PGCs) in a culture medium comprising leukemia inhibitory factor (LIF), basic fibroblast growth factor (bFGF), stem cell factor (SCF) and insulin-like growth factor (IGF), and avian serum; transferring the PGCs into a recipient avian embryo; allowing the recipient avian to develop into a bird; and selecting for chimeric avians which express the PGC phenotype.

In various embodiments of this aspect of the invention, the avian embryos may be turkey or chicken embryos. In the embodiments of this aspect of the invention, the PGCs may be injected into the dorsal aorta and/or marginal vein of a recipient avian embryo or into recipient blastoderms.

In an advantageous embodiment of this aspect of the invention, the culture medium may comprise a medium, about 10% fetal bovine serum, about 5% chicken serum, about 2 mM L-glutamine, about 1% antibiotic, about 0.13 mM 2-β-mercaptoethanol, about I U/μl leukemia inhibitory factor (LIF), about 40 pg/μl basic fibroblast growth factor (bFGF), about 80 pg/μl stem cell factor (SCF) and about 60 pg/μl insulin-like growth factor (IGF).

Another aspect of the invention encompasses a culture which may comprise purified isolated avian PGCs contained in a culture medium which may comprise growth factors in amounts that allow PGCs to be maintained in a viable state for at least fourteen days in tissue culture, wherein the culture medium may comprise leukemia inhibitory factor (LIF), basic fibroblast factor (bFGF), stem cell factor (SCF), insulin-like growth factor (IGF) and avian serum.

In one embodiment of this aspect of the invention, the culture may comprise a medium, about 10% fetal bovine serum, about 5% chicken serum, about 2 mM L-glutamine, about 1% antibiotic, about 0.13 mM 2-β-mercaptoethanol, about 1 U/μl leukemia inhibitory factor (LIF), about 40 pg/μl basic fibroblast growth factor (bFGF), about 80 pg/μl stem cell factor (SCF) and about 60 pg/μl insulin-like growth factor (IGF). In the embodiments of this aspect, the avian PGCs may be chicken or turkey PGCs.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

Following longstanding law convention, the terms “a” and “an” as used herein, including the claims, are understood to mean “one” or “more”.

The present invention improves on previous avian PGC culturing media and methods such as that disclosed in U.S. Pat. No. 6,156,569 to Ponce de Leon et al. incorporated herein by reference in its entirety. The culture medium of the present invention provides for an improved rate of cell growth and a higher percentage of cells having the desired PGC phenotype compared to other media such as that previously disclosed in U.S. Pat. No. 6,156,569 used under similar growth conditions. In particular, avian PGCs, advantageously Gallinacea PGCs, and most advantageously chicken PGCs can be grown at a faster rate and maintained in tissue culture for prolonged periods to at least about 50 days by the use of the novel culture medium may contain at least four growth factors, leukemia inhibiting factor (LIF), stem cell factor (SCF), insulin-like growth factor (IGF) and basic fibroblast growth factor (bFGF) and avian serum which has not previously incorporated into such media. Advantageously, the four growth factors may be at concentrations that are significantly elevated when compared to PGC culture media known in the art,

In general, the culturing method of the present invention encompasses, but may not be limited to, the steps of (i) isolating PGCs from donor avian embryos and (ii) culturing the isolated avian PGCs in a culture medium comprising amounts of LIF, bFGF, SCF, IGF and chicken serum effective to promote their proliferation for a prolonged time, i.e., at least 25 days, in tissue culture. Prolonged periods, as defined above, refers to a culture period 25 days or longer, advantageously, at least about 50 days and most advantageously indefinitely.

It is contemplated that the media of the present invention may also be useful for the culturing of other types of isolated avian cells including, but not limited to, embryonic stem (ES) cells, hematopoietic cells and the like and advantageously chicken ES cells. It is futher contemplated that the media of the present invention may be useful for the culturing of ES cells and PGCs isolated from genera other than just Gallus such as, but not limited to, turkeys. In such cases, it is anticipated that the avian serum may be obtained from the same genus of bird as the cells themselves. For example, chicken cells may be cultured in media containing chicken serum, turkey cells may be cultured in media containing turkey serum and so forth. However, it is further contemplated that serum from one avian genus may be used in culture media in which cells from a different genus are cultured such that, for example, chicken cells may be cultured in media containing turkey serum and vice versa.

Methods for the isolation of primordial germ cells from donor avian embryos have been reported in the literature and can be effected by one skilled in the art. (See, e.g., JP 924997 published Sep. 7, 1993 Pub. No. 05-227947; Chang et al., (1992) Cell Biol. Int., 19: 143-149; Naito et al., (1994) Mol. Reprod. Devel., 39: 153-161; Yasuda et al., (1992) J. Reprod. Fert., 96: 521-528; and Chang et al., (1992) Cell Biol. Int. Reporter, 16(9): 853-857, all of which are incorporated by reference in their entirety therein).

One protocol suitable for use in the present invention is to isolate avian PGCs from chicken eggs that have been incubated for about 53 hours (stage 12-14 of embryonic development), remove embryos therefrom, collect embryonic blood from the dorsal aorta thereof, and transfer the blood to suitable cell culture medium (α-MEM medium). These PGCs may then be purified by Ficoll density centrifugation, and resuspended in the growth factor-containing culture medium of the present invention. However, as discussed above, other methods for isolating PGCs are known and may alternatively be used.

The isolated PGCs may then be counted and separated manually (e.g., using a pipette). Thereafter, PGCs collected from multiple avian embryos may be pooled (to increase total PGC numbers) and incubated in the growth factor and avian serum-containing medium of the present invention.

This culture medium, hereinafter referred to as “complete” medium contains LIF, bFGF, SCF, IGF, and avian serum in a base medium as well as other substituents typically contained in PGC and embryonic stem cell medium. An advantageous embodiment, therefore, of the subject “complete” medium may include, but is not limited to, α-MEM, a well known commercially available cell growth medium base to which has been added the above four growth factors and avian serum. One embodiment of the medium, for example, includes with α-MEM about 10% fetal calf serum (FBS), 2 mM L-glutamine, 1% antibiotic/antimitotic, 0.13 mM 2-β-mercaptoethanol, about 1 U/μl of LIF, about 40 pg/μl of bFGF, about 60 pg/μl of IGF and about 80 pg/μl of SCF and about 5% chicken serum.

It is also contemplated that the amounts of the growth factors may be varied and while still maintaining proliferation of avian PGCs and ES cells. However, addition of the growth factors to the concentrations herein disclosed advantageously serves to prolong viable cultures and to significantly accelerate the rate of cell proliferation.

The media of the present invention may use as the base medium α-MEM, a well-known commercially available tissue culture medium. However, it is anticipated that other media bases such as DMEM, BME, CMRL Medium, F-10, Glasgow Minimal essential medium, Iscove's Modified Dulbecco's Medium, Medium 199, Minimal Essential Medium and Modified Eagle Medium and the like as supplied, for example by Invitrogen Corp. may be substituted therefor, provided that the four essential growth factors and avian serum are also present. It is also contemplated to be within the scope of the present invention for media to not include fetal calf serum, or to have fetal calf serum at substantially reduced concentrations. However, all media of the present invention will include avian serum, such as chicken serum.

While cultured PGCs have been grown in the absence of feeder cells, it is further contemplated that feeder cells may also be useful. In particular, the use of fibroblasts, advantageously avian fibroblasts, and most advantageously Gallinacea fibroblasts, and still more advantageously chicken fibroblasts, will provide for the maintenance of PGCs in tissue culture provided that the four essential growth factors LIF, IGF, SCF and b-FGF, and avian serum are present in the medium base. Feeder cells may be usefully transfected with genes encoding these growth factors, thereby eliminating the need for the exogenous addition of these factors during culturing. Essentially, the cells will provide a continual source of these growth factors. This may be achieved by placing these growth factor genes under control of constitutive strong promoter and also sequences that provide for the secretion thereof, thereby making these growth factors available to cultured PGCs. Suitable eukaryotic promoters include CMV immediate early, HSV thymidine kinase, early and late SV40, LTRs from retrovirus, and mouse metallothionein-1. Selection of the appropriate vector and promoter is well within the level of ordinary skill in the art.

The amounts of the growth factors refer to relative amounts thereof effective to enable the culturing of avian PGCs, advantageously Gallinacea PGCs, and most advantageously chicken or turkey PGCs, for prolonged periods in tissue culture at accelerated growth rates compared with currently used media.

Advantageously, the relative amounts of these growth factors may fall within the following ranges: LIF, about 0.00625 U/μl to about 5 U/μl, and most advantageously about 0.25 to about 3 U/μl; IGF, about 0.5625 pg/μl to about 150 pg/μl, more advantageously about 20 pg/μl to about 150 pg/μl and most advantageously about 0.5625 pg/μl to about 1.125 pg/μl; SCF about 4.0 pg/μl to about 400 pg/μl, more advantageously about 4.0 pg/μl to about 200 pg/μl and most advantageously about 40 pg/μl to about 200 pg/μl; and bFGF about 0.25 pg/μl to about 100 pg/μl, and most advantageously about 20 pg/μl to about 100 pg/μl.

However, it is expected that these preferred ranges may vary, e.g., if α-MEM is substituted by another growth medium such as DMEM, BME, CMRL Medium, F-10, Glasgow Minimal essential medium, Iscove's Modified Dulbecco's Medium, Medium 199, Minimal Essential Medium and Modified Eagle Medium and the like as supplied, for example by Invitrogen Corp. and if other types of avian PGCs are cultured. One of skill in the art will be able to adjust the concentrations of the growth factors according to the type of cells cultured and the medium components selected by routine experimentation that will provide ranges of concentration of the various components and allow determination of the relative growth rates and survival rates of cultured PGCs to select the optimum conditions.

PGCs grown and maintained in the avian serum-containing medium of the present invention can be maintained for long periods in culture with the successful production of chimeric avians therefrom. Cells cultured for up to about 50 days have been tested for their ability to effectively colonize avian embryonic gonads and produce chimeric birds. However, it is expected that these cells can be cultured indefinitely, with retention of the ability to produce chimeric birds.

Methods for using PGCs to produce chimeras are known in the art. PGCs may be transferred into recipient avian embryos according to the methods disclosed in the examples that follow. Thereafter, successful chimera production is evaluated based on migration and colonization of PGCs in the gonads, retention of PGC phenotype, or by evaluating for the presence of donor PGCs in gonads after hatching and breeding.

For example, genotypes may be selected that may have easily followed phenotypes, such as feather coloration. Donor birds may be white broiler type and recipient birds may be black-feathered birds, respectively, having specific potential genotypes. The putative chimeras would be black feathered and produce black/white progeny when mated with black birds. Thereby, successful chimeras can be demonstrated based on the production of black/white feathered progeny produced after mating the putative chimeric bird with another black-feathered bird.

However, the subject method should be applicable for introducing any desired trait by chimerization. This will, of course, depend on the genotypic properties of the transferred PGCs.

As discussed, a significant application of the subject PGCs, which can be maintained in culture for long periods, is for the production of chimeric avians bearing, for example, a heterologous nucleic acid that may be expressed by at least cells of the chimera. This can be accomplished by introducing a desired DNA sequence into the cultured PGCs. Means for introducing DNAs into recipient cells are known and include lipofection, transfection, microinjection, transformation, microprojectic techniques, etc.

Advantageously, a DNA will be introduced that encodes a desired gene, e.g., therapeutic polypeptide, growth factor, enzyme, etc., under the regulatory control of sequences operable in avians. Advantageously, these regulatory sequences will be of eukaryotic origin, most advantageously avian, e.g., chicken regulatory sequences. Promoters operable in avian cells, e.g., derived from avian genes or viruses are known in the art such as, but not limited to, the promoters for avian ovalbumin, ovomucoid, ovotransferrin.

Elements for the expression of the polynucleotide or polynucleotides are advantageously present in an inventive vector. In minimum manner, this comprises, consists essentially of, or consists of an initiation codon (ATG), a stop codon and a promoter, and optionally also a polyadenylation sequence for certain vectors such as plasmid and certain viral vectors, e.g., viral vectors other than poxviruses. When the polynucleotide encodes a polyprotein fragment, advantageously, in the vector, an ATG is placed at 5′ of the reading frame and a stop codon is placed at 3′. Other elements for controlling expression may be present, such as enhancer sequences, stabilizing sequences and signal sequences permitting the secretion of the protein.

A DNA “coding sequence” or a “nucleotide sequence encoding” a particular protein, is a DNA sequence which is transcribed and translated into a polypeptide in vitro or in vivo when place under the control of appropriate regulatory elements. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence.

DNA “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated. A control element, such as a promoter, “directs the transcription” of a coding sequence in a cell when RNA polymerase will bind the promoter and transcribe the coding sequence into mRNA, which is then translated into the polypeptide encoded by the coding sequence.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

A “host cell” is a cell that has been transformed, or is capable of transformation, by an exogenous nucleic acid molecule.

A cell has been “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cells containing the

Initially, a stable cell line that produces the desired protein will be isolated and used for chimera production. Also, it is desirable that the introduced DNA contains a marker DNA, the expression of which is easily detected, to more easily identify cells containing the inserted DNA. Such selectable markers are well known and include, but are not limited to, β-lactamase, β-galactosidase, neomycin phosphotranspherase, zeomycin resisance, blasticidin resistance, methotrexate resistance, tetracycline resistance, chloramphenicol, thymidine kinase and the like.

Injection of the resultant transgenic PGCs into avian embryos will then result in the production of transgenic chimeric avians. Advantageously, the desired protein will then be recovered from the eggs of these transgenic avians, thereby providing a continual supply of the protein. Alternatively, the protein can be recovered from chimeric birds directly, e.g., isolated from the systemic circulatory system.

Heterologous protein(s) produced from avian cells in accordance with the present invention can be isolated from the medium in which the cells are cultured using any of a variety of art-recognized techniques. Dialysis of the medium against dilute buffer or a superabsorbant material, followed by lyophilization, can be employed to remove the bulk of the low molecular weight components of the medium and to concentrate the heterologous protein. Alternatively, ultrafiltration or precipitation by saturation with salts such as sodium or ammonium sulfate can be used.

Once obtained in concentrated form, any standard technique, such as preparative disc gel electrophoresis, ion-exchange chromatography, gel filtration, size separation chromatography, isoelectric focusing and the like may be used to purify, isolate, and/or to identify the heterologous protein. Those skilled in the art may also readily devise affinity chromatographic, means of heterologous protein purification, especially for those instances in which a binding partner of the heterologous protein is known, for example, antibodies.

Those skilled in the art will recognize, or be able to ascertain using no more than routine experimentation, many equivalents to the specific embodiments of the invention described herein. Such equivalents are intended to be encompassed by the following claims.

One aspect of the invention, therefore, provides a modified culture medium for the proliferation and maintenance of avian primordial germ cells, the medium comprising in a medium base leukemia inhibitory factor (LIF), basic fibroblast growth factor (bFGF), stem cell factor (SCF) and insulin-like growth factor (IGF), and avian serum.

In the various embodiments of the invention, the avian serum may be present in the medium at a concentration of between about 0.5% v/v and about 10% v/v. In certain embodiments, the avian serum may be present in the medium at a concentration of between about 1% v/v and about 6% v/v. In one advantageous embodiment of the invention, the avian serum may be present in the medium at a concentration of about 5% v/v. In various embodiments of the invention, the avian serum may be present in the medium at a concentration of about 1% v/v.

In the various embodiments of the invention, the avian serum may be from any avian source. In a particularly advantageous embodiment of the invention, however, the avian serum may be chicken serum or turkey serum and most advantageously, chicken serum.

In the various embodiments of the invention, the culture medium for the proliferation and maintenance of avian primordial germ cells contains in a medium base leukemia inhibitory factor (LIF) between about 0.00625 U/μl and about 5 U/μl, basic fibroblast growth factor (bFGF) between about 0.25 pg/μl and about 100 pg/μl, stem cell factor (SCF) between about 4 pg/μl and about 200 pg/μl and insulin-like growth factor (IGF) between about 0.5625 pg/μl and about 150 pg/μl.

In some embodiments of this aspect of the invention, the leukemia inhibitory factor (LIF) is between about 0.25 U/μl and about 3 U/μl, basic fibroblast growth factor (bFGF) is between about 25 pg/μl and about 100 pg/μl, stem cell factor (SCF) is between about 40 pg/μl and about 200 pg/μl and insulin-like growth factor (IGF) is between about 10 pg/μl and about 150 pg/μl.

In an advantageous embodiment of the invention, the leukemia inhibitory factor (LIF) is about 1 U/μl, basic fibroblast growth factor (bFGF) is about 40 pg/μl, stem cell factor (SCF) is about 80 pg/μl and insulin-like growth factor (IGF) is about 60 pg/μl.

In the various embodiments of the invention, the culture medium further comprises 2-β-mercaptoethanol having a concentration of between about 0.05 mM and about 50 mM. In one embodiment of the invention, the 2-β-mercaptoethanol has a concentration of between about 0.05 mM and about 0.25 mM. In one advantageous embodiment, the 2-β-mercaptoethanol has a concentration of about 0.13 mM.

This aspect of the invention also encompasses a culture medium for the proliferation and maintenance of avian primordial germ cells comprising a medium, fetal bovine serum, avian serum, L-glutamine, an antibiotic, 2-β-mercaptoethanol, leukemia inhibitory factor (LIF), basic fibroblast growth factor (bFGF), stem cell factor (SCF) and insulin-like growth factor (IGF).

In a particularly advantageous embodiment of the invention, the culture medium comprises a medium, about 10% fetal bovine serum, about 5% chicken serum, about 2 mM L-glutamine, about 1% antibiotic, about 0.13 mM 2-β-mercaptoethanol, about 1 U/μl leukemia inhibitory factor (LIF), about 40 pg/μl basic fibroblast growth factor (bFGF), about 80 pg/μl stem cell factor (SCF) and about 60 pg/μl insulin-like growth factor (IGF).

Another aspect of the invention encompasses a culturing method for the proliferation and maintenance of avian primordial germ cells for periods of at least 25 days in tissue culture, the method comprising the steps of isolating a pure population of primordial germ cells from a desired avian, and culturing the isolated, pure population of primordial germ cells (PGCs) in a culture medium comprising leukemia inhibitory factor (LIF), basic fibroblast growth factor (bFGF), stem cell factor (SCF) and insulin-like growth factor (IGF), and avian serum.

In the various embodiments of this aspect of the invention, the avian PGCs may be obtained from an avian of the genus Gallinacea. In one embodiment of the invention, the PGCs may be chicken PGCs or turkey PGCs.

Yet another aspect of the invention encompasses a method of producing chimeric avians, the method comprising the steps of isolating a pure population of primordial germ cells from a desired avian; maintaining the isolated pure population of primordial germ cells (PGCs) in a culture medium comprising leukemia inhibitory factor (LIF), basic fibroblast growth factor (bFGF), stem cell factor (SCF) and insulin-like growth factor (IGF), and avian serum; transferring the PGCs into a recipient avian embryo; allowing the recipient avian to develop into a bird; and selecting for chimeric avians which express the PGC phenotype.

In various embodiments of this aspect of the invention, the avian embryos may be turkey or chicken embryos. In the embodiments of this aspect of the invention, the PGCs may be injected into the dorsal aorta and/or marginal vein of a recipient avian embryo or into recipient blastoderms.

In an advantageous embodiment of this aspect of the invention, the culture medium comprises a medium, about 10% fetal bovine serum, about 5% chicken serum, about 2 mM L-glutamine, about 1% antibiotic, about 0.13 mM 2-β-mercaptoethanol, about 1 U/μl leukemia inhibitory factor (LIF), about 40 pg/μl basic fibroblast growth factor (bFGF), about 80 pg/μl stem cell factor (SCF) and about 60 pg/μl insulin-like growth factor (IGF).

Yet another aspect of the invention encompasses a culture consisting essentially of purified isolated avian PGCs contained in a culture medium which comprises growth factors in amounts that allow PGCs to be maintained in a viable state for at least fourteen days in tissue culture, wherein the culture medium comprises leukemia inhibitory factor (LIF), basic fibroblast factor (bFGF), stem cell factor (SCF), insulin-like growth factor (IGF) and avian serum.

In one embodiment of this aspect of the invention, the culture comprises a medium, about 10% fetal bovine serum, about 5% chicken serum, about 2 mM L-glutamine, about 1% antibiotic, about 0.13 mM 2-β-mercaptoethanol, about 1 U/μl leukemia inhibitory factor (LIF), about 40 pg/μl basic fibroblast growth factor (bFGF), about 80 pg/μl stem cell factor (SCF) and about 60 pg/μl insulin-like growth factor (IGF). In the embodiments of this aspect, the avian PGCs may be chicken or turkey PGCs.

It should be understood that the present invention is not limited to the specific compositions or methods described herein and that any composition having a formula or method steps equivalent to those described falls within the scope of the present invention. Preparation routes of the composition and method steps are merely exemplary so as to enable one of ordinary skill in the art to make the composition and use it according to the described process and its equivalents. It will also be understood that although the form of the invention shown and described herein constitutes advantageous embodiments of the invention, it is not intended to illustrate all possible forms of the invention. The words are words of description rather than of limitation. Various changes and variations may be made to the present invention without departing from spirit and scope of the invention.

The invention is illustrated by the following non-limiting examples:

### EXAMPLE 1

### Experimental Materials and Methods

(a) Animals. A commercial strain of broiler type chickens has been used as donors of PGCs to develop the long term PGC culture system and as recipient embryos.

(b) Extraction of PGCs. Stage 13 to 14 embryos were selected for PGC extraction. PGCs were collected from the dorsal aorta with a fine micropipette as described by Naito et al., (1994) Mol. Reprod. Dev., 37: 167-171. PGCs from 20 embryos were pooled in Hanks' solution supplemented with 10% fetal bovine serum and concentrated by Ficoll density gradient centrifugation. PGCs were counted and distributed in 10 μl drops of culture medium on microslides or plastic culture plates at about 100 PGCs per drop. Culture drops were overlaid with sterile light mineral oil. Medium was renewed by washing with 5 μl volumes three times with fresh medium from under the oil overlay. Cultured cells, as either monolayers or as cell clumps, were treated with Accutase™ (Innovative Cell Technologies, Inc, San Diego, Calif.) according to manufacturer's instructions.

(c) Injection of PGCs Into Recipient Embryos. Stage 14-15 embryos were used as recipient embryos. After the recipient egg was positioned horizontally under a dissecting scope, time was allowed for the developing embryo to position itself on the upper side of the resting egg. A small hole was pierced into the air space of the egg to lower the internal pressure of the egg and prevent leakage. A small, about 10 mm “window” or less was opened on the ventral surface of the egg with a fine forceps. The embryo was brought close to the surface by adding about. 1 ml of PBS/4% antibiotic/antimitotic injected through the hole to bring the embryo up until it was slightly less than flush with the eggshell window.

To inject the PGCs, a 30 μm pipette was beveled and then pulled using a microforge to form a fine point with polished edges. After accommodating the embryo to visualize its heart, the marginal vein and/or dorsal aorta could be easily identified. Injections were made directly into the embryo or preferably into the aortic chamber of the heart formed by the merging of blood vessels and which represents an easier target than the somewhat smaller marginal veins. About two hundred donor PGCs in 2 μl of media containing 0.04% trypan blue were taken into a micropipette. PGCs were injected into the dorsal aorta of the recipient embryo. Trypan blue, an inert cell dye, allowed visualization of the PGC suspension when it was being delivered. After injection, the eggshell opening was closed with surgical tape and reinforced with paraffin. Eggs were maintained for 24 hours under surveillance in a humidified CO₂ incubator and later transfer to a regular incubator until hatching.

(d) Viable Fluorescent Staining of PGCs. To evaluate the success of transfers and/or the ability of PGCs to migrate to the gonads, PGCs were stained with the lipophilic carbocyanine DiI fluorescent stain. Embryos were collected after 24 hours of transfer, placed on a petri dish and observed under an inverted microscope equipped for epi-fluorescent analysis.

### EXAMPLE 2

### Culture Medium

A complete cell culture medium had the following composition: α-MEM, 10% fetal calf serum, 5% chicken serum, 2 mM L-glutamine, 1% antibiotic/antimitotic, 0.13 mM 2-β-mercaptoethanol, 1 U/μl of leukemia inhibitory factor (LIF), 40 pg/μl of basic fibroblast growth factor (b-FGF), 60 pg/μl of insulin like growth factor (IGF) and 80 pg/μl of stem cell factor (SCF). Either human or mouse (recombinant) LIF is suitable for use in the medium.

To prepare the chicken serum, blood was collected from the necks of adult birds and centrifuged at 1000 rpm for about 15 mins. to remove the blood cells. The serum supernatant was transferred to fresh centrifuge tubes and respun to remove any remaining blood cells and debris. The serum was then filtered through a 0.22 μm filter and stored overnight at 4° Celsius. This could result in a precipitate forming, which was removed by refiltering. The clarified serum was then aliquoted and stored frozen at −20° Celsius. Alternatively a commercial source of chicken serum may be used such as Sigma Cat No. C5405.

Medium changes were carried out every other day by removing 5 μl of medium from 10 μl cell cultures and adding 5 μl of fresh medium containing 2× concentration of the growth factors, repeating three times. This assumed that growth factors would be labile after some period of continuous culture. However, the net result can be that the concentrations of growth factors are doubled. Hence, the final medium briefly could contain the following growth factor concentrations: 2 U/μl of leukemia inhibitory factor (LIF), 80 pg/μl of basic fibroblast growth factor (bFGF), 120 pg/μl of insulin like growth factor (IGF) and 160 pg/μl of stem cell factor (SCF).

Five microliters of the culture medium (now old and new medium combined) was then removed and replaced with 5 ml of new medium with the growth factors at 1× concentration. This step was repeated twice more.

The ranges of growth factor concentrations described herein promote the maintenance and proliferation of PGCs in continuous culture. However, PGCs may survive and proliferate better at the highest end of the described growth factor concentrations. None of the growth factors alone, at any of the concentrations studied, was able to sustain PGCs in vitro without differentiation. Combinations of two and three growth factors were also tested with little success. All of the factors described above (LIF BFGF, IGF and SCF) may be required for long term culture of PGCS.

### EXAMPLE 3

### Culturing of PGCs

Avian PGCs were isolated from chicken eggs that had been incubated for about 53 hours (stage 12-14 of embryonic development), embryos were removed, embryonic blood was collected from the dorsal aorta, and the blood transferred to α-MEM-based suitable cell culture medium. These PGCs were then be purified by Ficoll density centrifugation, and resuspended in growth factor- and avian (chicken) serum-containing culture medium.

The isolated PGCs were then counted and separated manually (e.g., using a pipette). Thereafter, PGCs collected from multiple avian embryos were be pooled (to increase total PGC numbers) and incubated in the growth factor- and avian serum-containing medium.

After collection, PGCs were recognized by their size and by the presence of lipid droplets in their cytoplasm. At about 48 hours after collection, PGCs clumped together and started dividing as evidenced by the growth in size of the clump and the number of cells observed after trypsin dissociation of the clump. Only PGCs that formed clumps survived; all others died. Generally, a culture was started with from about 100 to about 500 PGCs per 10 μl culture, most generally about 250 PGCs per starting inoculum, and would end up with an average of 600 to 800 PGCs within seven days. However, as indicated above, these PGCs maintained their ability to migrate to the gonads.

Avian PGCs formed clumps within the first 2-4 days of culture, with the clumps becoming larger as the cells within continue to divide. With the addition of avian serum, there was an increase in the proportion of cells that are viable, with a reduction or elimination of visible dead cells and cellular debris. In some cultures, the appearance of slowly dividing epithelial-like cells spread through out the culture was recorded. Although such epithelial-like cells may not be necessary for the improved growth of PGCs, it has been observed that they were more often found in cultures having less cell debris.

After 19 days in culture, the amount of cells counted indicated a growth rate in the medium containing chicken serum as at least three-fold greater than in medium lacking chicken serum (as described, for example, in U.S. Pat. No. 6,156,569) under comparable growth conditions. In addition, it was found that with the medium containing chicken medium, approximately 25% more of the cultured cells were positive for the markers VASA and stage-specific embryonic antigen (SSEA-1), two common markers for PGCs (see, for example, Tsunckawa et al., (2000) Development 127: 2741-2750, and Jung et al., (2005) Stem Cells 23: 689-98). For the staining method, see Example 5 below). Additionally, cells cultured in this media through 45 days remained positive for Periodic Acid Schiff staining, another common characteristic of PGCs.

At weeks 1, 2 and 3, clumps have been dissociated, stained with a vital dye DiI and transferred into recipient embryos. At all three time-points cells were found in the gonads of some of the recipient embryos. The number of cells and the number of embryos showing stained PGCs in the gonads was inversely proportional to the age of the PGCs culture.

Chicken PGCs are positive for periodic acid Schiff staining (PAS). Their phenotype was evaluated by transferring cells to recipient embryos and evaluating their presence in the gonads of the developing embryo. This method required culturing the PGCs in 100 μg/ml DiI in an α-MEM medium and rinsing prior to transfer to recipient embryos. Twenty-four hours post-transfer, recipient embryos were removed and placed under an inverted microscope. DiI labeled cells observed in the gonads were interpreted as successful PGC migration to the gonads and confirmation of retention of PGC characteristics. A second method to evaluate the retention of the PGC phenotype was pursued by letting recipient embryos go to hatching and then evaluate the presence of donor PGCs in their gonads after breeding.

At least one of these embryos was chimeric as demonstrated by progeny testing. PGCs that had been maintained in culture for 46-48 days were also transferred to recipient embryos. Based on DiI staining of PGCs and under the culture conditions described above, PGCs originating from 46-48 day old continuous cultures migrated to the gonads of recipient embryos alter injection.

### EXAMPLE 4

### Long-Term Cultures Beyond 25 Days

Cells cultured for up to 43-48 days and having been passaged, were stained with the vital dye DiI and injected into the circulation of embryos at about 56 hours of incubation. The recipients were analyzed by either observing the area of the gonadal ridge at 70 hour incubation point, or the gonads were extracted at the 5.5 day incubation point. At both stages of embryonic development, labeled cells were identified indicating viability and incorporation into developing embryonic tissue.

### EXAMPLE 5

### Immunofluorescent Labeling of Cells on Microslides

Cells were air-dried on standard glass microslides and heat-fixed by holding over an alcohol lamp until condensation dissipated. At this point slides could be stored at 4° Celsius. Slides were then fixed by immersion in 4% paraformaldehyde 4° Celsius for 15 mins. and immersed in blocking solution (1% normal goat serum in PBST) at 4° Celsius for 30 mins. Excess liquid was removed from the slide by briefly laying the edge of the slide on absorbent paper and wiping the undersurface of slide. Slides were then placed horizontally with the cell samples facing up.

The cell sample fixed to the microslide was covered with 75-200 μl of a primary antibody solution and a cover slip placed over sample while making sure that no air bubbles were in contact with the sample. Primary antibody solutions were diluted in block solution as follows: SSEA-1 diluted between 1:250 and 1:1000 (1;500 of antibody #MC-480, Developmental Studies Hybridoma Bank, University of Iowa); Vasa or DAZL between 1:100 and 1:1000. It was possible to mix SSEA1 with either VASA or DAZL for simultaneous use. Samples could then be left at room temperature for 1-2 hours or at 4° Celsius overnight.

The cover slip was removed and discarded ad the slide washed 3× for 5 mins. each in PBST (0.1% TWEEN 20 in PBS). Excess liquid was removed from the slide by briefly laying the edge of the slide on absorbent paper and wiping the undersurface of slide. From this point on in the procedure, the samples were protected from light.

The samples were then covered with 75-200 μl of a secondary antibody solution, covered with a cover slip while ensuring no air bubbles were in contact with samples and incubated at room temperature for 1 hour. The secondary fluorescent antibody was diluted 1:500 in block solution. For SSEA-1 primary antibody, anti-mouse IgM was used. With anti-VASA or anti-DAZL antibody, anti-rabbit IgG was the secondary antibody. Either FITC or TRITC fluorescent label was be used, but both were used when anti-SSEA-1 antibody was mixed with either anti-VASA or anti-DAZL antibody. The samples were then washed 3× for 5 minutes in PBST and excess liquid removed as described above.

The sample was then stained in DAPI (Sigma Cat No. D8417 at 100 μg/ml in water diluted 1:1000 in 1×PBS) at room temperature for 10 mins. and rinsed in PBST. One drop of DABCO (prepared by adding 0.233 g DABCO, Sigma Cat. No. D2522, to 800 μl distilled water and 200 μl of 1M Tris, pH 8.0, and then added to 9 ml glycerol. The stock solution was stored at −70° Celsius.

Having thus described in detail advantageous embodiments of the present invention, it is to be understood that the invention defined by the above paragraphs is not to be limited to particular details set forth in the above description as many apparent variations thereof are possible without departing from the spirit or scope of the present invention.

1. A culture medium comprising leukemia inhibitory factor (LIF), basic fibroblast growth factor (bFGF), stem cell factor (SCF) and insulin-like growth factor (IGF), and avian serum.

2. The culture medium according to claim 1, wherein the avian serum is present in the culture medium at a concentration of between about 0.5% v/v and about 10% v/v, or wherein the avian serum is present in the medium at a concentration of between about 1% v/v and about 6% v/v, or wherein the avian serum is present in the medium at a concentration of about 5% v/v, or wherein the avian serum is present in the medium at a concentration of about 1% v/v.

3. The culture medium according to claim 1, wherein the avian serum is chicken serum.

4. The culture medium according to claim 1, wherein the leukemia inhibitory factor (LIF) is between about 0.00625 U/μl and about 5 U/μl, basic fibroblast growth factor (bFGF) is between about 0.25 pg/μl and about 100 pg/μl, stem cell factor (SCF) is between about 4 pg/μl and about 200 pg/μl and insulin-like growth factor (IGF) is between about 0.5625 pg/μl and about 150 pg/μl.

5. The culture medium according to claim 1, wherein the leukemia inhibitory factor (LIF) is between about 0.25 U/μl and about 3 U/μl, basic fibroblast growth factor (bFGF) is between about 25 pg/μl and about 100 pg/μl, stem cell factor (SCF) is between about 40 pg/μl and about 200 pg/μl and insulin-like growth factor (IGF) is between about 10 pg/μl and about 150 pg/μl.

6. The culture medium according to claim 1, wherein the leukemia inhibitory factor (LIF) is about 1 U/μl, basic fibroblast growth factor (bFGF) is about 40 pg/μl, stem cell factor (SCF) is about 80 pg/μl and insulin-like growth factor (IGF) is about 60 pg/μl.

7. The culture medium according to claim 1, further comprising between about 0.05 mM and about 50 mM 2-β-mercaptoethanol, or between about 0.05 mM and about 0.25 mM 2-β-mercaptoethanol, or about 0.13 mM 2-β-mercaptoethanol.

8. A culture medium comprising a medium base, fetal bovine serum, avian serum, L-glutamine, an antibiotic, 2-β-mercaptoethanol, leukemia inhibitory factor (LIF), basic fibroblast growth factor (bFGF), stem cell factor (SCF) and insulin-like growth factor (IGF).

9. The culture medium according to claim 8, comprising a medium base, about 10% fetal bovine serum, about 5% chicken serum, about 2 mM L-glutamine, about 1% antibiotic, about 0.13 mM 2-β-mercaptoethanol, about 1 U/μl leukemia inhibitory factor (LIF), about 40 pg/μl basic fibroblast growth factor (bFGF), about 80 pg/μl stem cell factor (SCF) and about 60 pg/μl insulin-like growth factor (IGF).

10. A method for maintaining avian primordial germ cells for periods of at least fourteen days in tissue culture comprising:
(i) isolating a pure population of primordial germ cells from a desired avian; and
(ii) culturing the isolated, pure population of primordial germ cells (PGCs) in a culture medium according to claim 1.

11. The method according to claim 10, wherein the avian PGCs are obtained from an avian of the genus Gallinacea.

12. The method according claim 11, wherein the PGCs are chicken PGCs or turkey PGCs.

13. A method of producing chimeric avians comprising:
(i) isolating a pure population of primordial germ cells from a desired avian;
(ii) maintaining the isolated, pure population of primordial germ cells (PGCs) in a culture medium according to claims 1;
(iii) transferring the PGCs into a recipient avian embryo;
(iv) allowing the recipient avian to develop into a bird, and
(v) selecting for chimeric avians that express the PGC phenotype.

14. The method according to claim 13, wherein the avian embryos are turkey or chicken embryos.

15. The method according to claim 13, wherein the PGCs are injected into the dorsal aorta and/or marginal vein of a recipient avian embryo or into recipient blastoderms.

16. The method according to claim 13, wherein culture medium comprises a medium base, about 10% fetal bovine serum, about 5% chicken serum, about 2 mM L-glutamine, about 1% antibiotic, about 0.13 mM 2-β-mercaptoethanol, about 1 U/μl leukemia inhibitory factor (LIF), about 40 pg/μl basic fibroblast growth factor (bFGF), about 80 pg/μl stem cell factor (SCF) and about 60 pg/μl insulin-like growth factor (IGF).

17. A culture comprising purified isolated avian PGCs contained in a culture medium which comprises growth factors in amounts sufficient to maintain said PGCs for at least fourteen days in tissue culture, wherein the culture medium comprises leukemia inhibitory factor (LIF), basic fibroblast factor (bFGF), stem cell factor (SCF), insulin-like growth factor (IGF) and avian serum.

18. The culture of claim 17, wherein the culture comprises a medium, about 10% fetal bovine serum, about 5% chicken serum, about 2 mM L-glutamine, about 1% antibiotic, about 0.13 mM 2-β-mercaptoethanol, about 1 U/μl leukemia inhibitory factor (LIF), about 40 pg/μl basic fibroblast growth factor (bFGF), about 80 pg/μl stem cell factor (SCF) and about 60 pg/μl insulin-like growth factor (IGF).

19. The culture of claim 18, wherein the avian PGCs are chicken or turkey PGCs.
